(19) United States
(12) Patent Application Publication
(10) Pub. No.: US 2024/0066227 A1
(43) Pub. Date: Feb. 29, 2024

(54) KEYED CONNECTORS FOR MEDICAMENT DELIVERY DEVICES

(71) Applicant: HASELMEIER AG, Gallen (CH)

(72) Inventor: Joachim KEITEL, Esslingen (DE)

(21) Appl. No.: 17/766,956

(22) PCT Filed: Oct. 1, 2020
(86) PCT No.: PCT/EP2020/077459

§ 371 Date: Apr. 6, 2022

Related U.S. Application Data
(60) Provisional application No. 62/912,461, filed on Oct. 8, 2019.

(51) Int. Cl. A61M 5/24

(52) U.S. Cl. CPC A61M 5/24; A61M 2005/2488

(57) ABSTRACT

A collection of two or more dose delivery mechanisms and two or more medicament containers includes a first dose delivery mechanism comprising a first keyed connector and a second dose delivery mechanism comprising a second keyed connector. A first holder having a distal end with a third keyed connector. A second holder having a distal end with a fourth keyed connector. The first keyed connector not capable of forming a connection with the fourth keyed connector and the second keyed connector not capable of forming a connection with the third keyed connector. The first keyed connector configured to engage and form a connection with the third keyed connector and the second keyed connector configured to engage and form a connection with the fourth keyed connector.

### CROSS-REFERENCE TO RELATED APPLICATION

This application is a U S. National Stage application of International Application No. PCT/EP2020/077459, filed Oct. 1, 2020, which claims priority to U.S. Provisional Application No. 62/912,461, filed Oct. 8, 2019, the contents of each of which are incorporated herein by reference in their entirety.

### BACKGROUND

### Field of the Invention

The present patent disclosure is generally directed to drug delivery devices and reservoirs, particularly reservoirs containing a medicament. More particularly, the present disclosure is directed to injection delivery devices having a medicament container assembly attached to a dose setting and delivery mechanism, and in particular to such medicament holder assemblies that have coding features that engage complimentary or cooperating coding features on the dose setting and delivery mechanisms to ensure the appropriate medicament container assembly is connected to the appropriate dose setting and delivery mechanism. In other words, the present disclosure provides mechanical solutions to prevent unwanted cross use of a medicament.

### BACKGROUND OF THE INVENTION

There are a number of medicament delivery devices on the market that are capable of automatically, semi-automatically or manually delivering doses of medicament. Of the known type of delivery devices, the “pen-type” injector is popular and is available in both reusable and disposable designs. Such devices are constructed with dose setting and delivery mechanisms that include a variety of inter-acting mechanical components to achieve desired functions, such as setting a dose, dose cancellation, and ultimately delivering the set dose. Such devices are typically designed for non-medically trained individuals to self-administer medicaments. Users of such devices include diabetics, where medication management and compliance, i. e. the degree to which a patient follows medical instructions and protocols, is often of extreme importance.

### SUMMARY

This disclosure is focused on medicament delivery devices that are reusable and semi-reusable, meaning that the dose setting and delivery mechanism is repeatedly used with new containers of medicament that replace each previously used container as they become empty.

Medicament containers or reservoirs such as ampoules, cartridges, or vials are generally known. Such reservoirs are especially used for medicaments that may be self-administered by a patient. For example, with respect to insulin, a patient suffering from diabetes can require a certain amount of insulin to either be injected via a pen type injection syringe or infused via a pump. With respect to certain known reusable pen type drug delivery devices, a patient loads a cartridge containing the medicament into a distal end of a cartridge holder. After the cartridge has been correctly loaded and the cartridge holder properly is connected to the dose setting mechanism, the user can then be called upon to select a dose of medicament. Multiple doses can be dosed from the cartridge. Where the drug delivery device comprises a reusable device, once the cartridge is empty, the cartridge holder is disconnected from the drug delivery device and the empty cartridge is removed and replaced with a new cartridge. Most suppliers of such cartridges recommend that the user dispose of the empty cartridges properly. Where the drug delivery device comprises a semi-disposable device, once the cartridge is empty, the user will dispose of both the cartridge and the cartridge holder together, which is typically provided as single assembly, meaning that the cartridge is sealed or otherwise permanently fixed within the holder. A new assembly (cartridge plus holder) is then attached to the reusable dose setting and delivery mechanism.

Such known self-administration systems that require the removal and reloading of empty cartridges have certain limitations. For example, in certain generally known systems, a user simply loads a new cartridge into a cartridge holder and there is no mechanism of ensuring the correct cartridge and/or medicament is loaded. That is, the drug delivery device does not have a mechanism for determining if the medicament contained in the cartridge is indeed the correct type of medicament to be administered by the patient. Alternatively, certain known drug delivery devices do not present a mechanism for determining if the correct type of medicament within the cartridge should be used with that particular drug delivery system. This potential problem could be exacerbated given that certain elderly patients, such as those suffering from diabetes, can have limited manual dexterity. Identifying an incorrect medicament is quite important, since the administration of a potentially incorrect dose of a medicament such as a short acting insulin in lieu of a long insulin could result in injury or even death.

Another concern that can arise with such disposable cartridges is that these cartridges are manufactured in essentially standard sizes and manufactured to comply with certain recognized local and international standards. Consequently, such cartridges are typically supplied in standard sized cartridges (e. g., 3 ml cartridges). Therefore, there can be a variety of cartridges supplied by a number of different suppliers and containing a different medicament, but they all can fit into a single cartridge holder that is to be attached to a drug delivery device. As just one example, a first cartridge containing a first medicament from a first supplier can fit into a second medical delivery device provided by a second supplier. As such, there exists the possibility that a user might be able to load an incorrect medicament and then dispense it through a drug delivery device without being aware that the medical delivery device was perhaps neither designed nor intended to be used with such a cartridge of medicament.

To minimize or completely alleviate such cross use of incorrect cartridges of medicament, the present disclosure is directed to medicament container holders that have coded or keyed connectors that match with corresponding coded connectors on the dose setting and delivery mechanism part of the drug delivery device. Using coded connectors on such holders allows the use of medicament reservoirs in the present disclosure such as an ampoule, a cartridge, a vial, or a pouch. Exemplary medical delivery devices include, but are not limited to syringes, pen type injection syringes, pumps, inhalers, or other similar injection or infusing devices that require at least one reservoir containing at least one medicament.

The disclosure presented below achieves the above-mentioned goals by providing an injector design that allows for more accurate evaluation of user compliance with medical treatment protocols, and provides an inherent safety feature preventing the misuse of a medicament.

As stated, this disclosure is directed to any number of medicament holders that are capable of accepting and holding containers of medicament provided the holder has a coded connector that matches a corresponding coded connector on the dose setting and delivery mechanism portion of the drug delivery device. For example, the holders of the present disclosure can be made available to a user in a configuration where the holder contains a medicament container that is permanently fixed within the holder. Stated differently, the container of medicament cannot be removed from the holder unless the holder is broken or damaged such that it would be inoperable with the intended corresponding dose setting and delivery mechanism, or for that matter any delivery mechanism.

Alternatively, the container of medicament could be removable from the coded holder so that an empty container could be replaced with a new full container. In such a situation it would be beneficial if the container itself contained a coding feature that would prevent an incorrect container from being inserted into the coded holder. The present disclosure is not directed to disposable drug delivery devices where the medicament container holder is permanently attached to the dose setting and delivery mechanism. For a disposable device once the container of medicament is empty, the entire delivery device is disposed of. In such a disposable device there is no mechanism to remove an empty container of medicament, resetting of the piston rod, or inserting a new filled container.

Preferably, the drug delivery device of the present disclosure is a pen-type injection device that is capable of variable, user settable, multiple doses from a single container of medicament, where the container is preferably a cartridge. Examples of such devices are described in U.S. Pat. No. 8,512,296, U.S. Pub. No. 2018/0001031 and U.S. Ser. No. 15/649,287, filed Jul. 13, 2017, the contents of each of these patent applications are fully incorporated by reference in this application. Such injection devices can be reusable so that the container, typically a cartridge, of medicament is replaceable through partial disassembly and resetting of the dose setting and delivery mechanism portion of the injection device, for example, by replacing an empty cartridge with a full cartridge and retracting a piston rod back into a dose setting mechanism. In a reusable device, it is necessary that the cartridge holder is removed from the proximal end of the dose setting mechanism and the used empty cartridge is replaced by a new full cartridge, and the cartridge holder is then reattached to the dose setting mechanism.

In one embodiment of the present disclosure there is presented a collection of injection devices made up of at least two or more dose setting and delivery portions of drug delivery device, i. e., the delivery mechanism portion, and two or more medicament containers. A first dose delivery mechanism has a proximal end comprising a first keyed connector and a second dose delivery mechanism has a proximal end comprising a second keyed connector. The first holder is configured to accept and hold a first medicament container containing a first medicament, where a distal end of the first holder comprises a third keyed connector. A second holder is configured to accept and hold a second medicament container containing a second medicament, where a distal end of the second holder comprises a fourth keyed connector and the second medicament is different than the first medicament.

The first keyed connector will not form a connection with the fourth keyed connector and the second keyed connector will not form a connection with the third keyed connector, and the first keyed connector can engage and form a connection with the third keyed connector and the second keyed connector can engage and form a connection with the fourth keyed connector.

The collection of dose delivery mechanisms and medicament containers of an embodiment of the present disclosure can be configured such that the first and second holders have an open proximal end configured to allow axial movement of a piston rod contained within one of the first or second dose delivery mechanisms such that the piston rod moves beyond the proximal end into the first holder when the first holder is attached to the first dose delivery mechanism. The first and second keyed connectors 10 can also each comprise a thread form, where the thread forms each have the same core diameter. Likewise, the thread forms each can have the same outer thread diameter. In some embodiments is preferable that the thread form of the first keyed connector is of opposite hand to the thread form of the second keyed connector and/or the thread form of the first keyed connector has a first pitch and the thread form of the second keyed connector has a second pitch. The first pitch and the second pitch can be different.

The thread form of the first keyed connector can also have a first thread width and the thread form of the second keyed connector has a second width. Additionally, the thread form of the first keyed connector can have a first thread geometry and the thread form of the second keyed connector can have a second geometry, where the first thread geometry is different than the second geometry. In some instances, the first and second keyed connectors each comprise a male thread form having the same core diameter and different outer diameters. Where a male thread is used on the first and second keyed connectors, each male thread form can have a different geometry and/or a different pitch. Additionally, the male thread form of the first keyed connector can have a hand opposite that of the male thread form of the second keyed connector.

Of course, there embodiments of the present disclosure where the first and second keyed connectors each are configured as a female male thread form.

These and other aspects of, and advantages with, the present disclosures will become apparent from the following detailed description of the present disclosure and from the accompanying drawings.

### BRIEF DESCRIPTION OF DRAWINGS

In the following detailed description of the present disclosure, reference will be made to the accompanying drawings.

FIG. 1 is a perspective illustration of a possible complete medicament delivery device containing the structural components of the present disclosure;

FIG. 2 shows a perspective illustration of the device of FIG. 1 where the cap is removed allowing attachment of a pen needle to the cartridge holder;

FIG. 3 is an exploded view of the device of FIGS. 1 and 2;

FIG. 4 is a cross-sectional side view of one embodiment of the present disclosure where the keyed connectors match and are partially engaged with each other;

FIG. 5A shows a side view of one possible keyed connector of the proximal end of a dose setting and delivery mechanism;

FIG. 5B shows a truncated perspective view of the keyed connector shown in FIG. 5A;

FIG. 6A shows a side view of another possible keyed connector of the proximal end of a dose setting and delivery mechanism;

FIG. 6B shows a truncated perspective view of the keyed connector shown in FIG. 6A;

FIG. 7A shows a side view of another possible keyed connector of the proximal end of a dose setting and delivery mechanism;

FIG. 7B shows a truncated perspective view of the keyed connector shown in FIG. 7A; and

FIG. 8 shows a truncated cross-sectional side view of two identical cartridge holders and two different dose setting and delivery mechanism housings each having two different keyed connectors.

### DETAILED DESCRIPTION

In the present application, the term “distal part/end” refers to the part/end of the device, or the parts/ends of the components or members thereof, which in accordance with the use of the device, is located the furthest away from a delivery/injection site of a patient. Correspondingly, the term “proximal part/end” refers to the part/end of the device, or the parts/ends of the members thereof, which in accordance with the use of the device is located closest to the delivery/injection site of the patient.

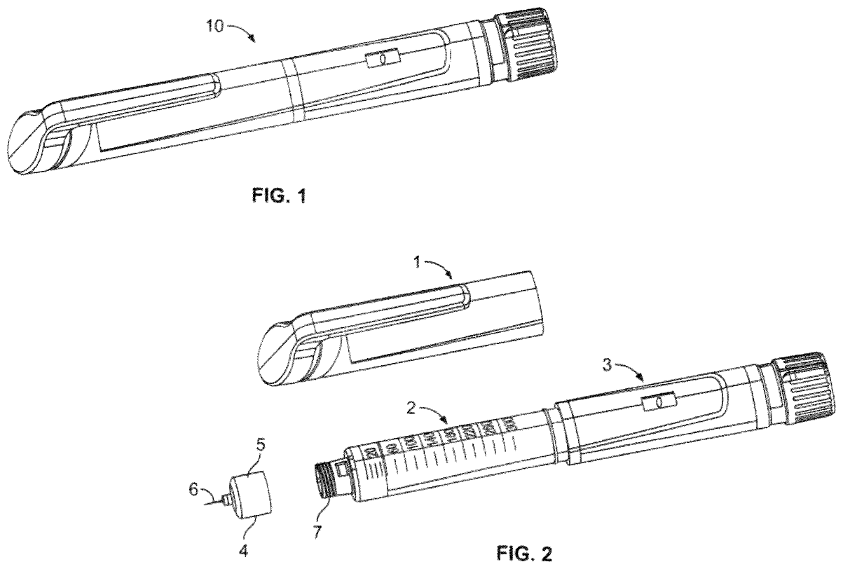

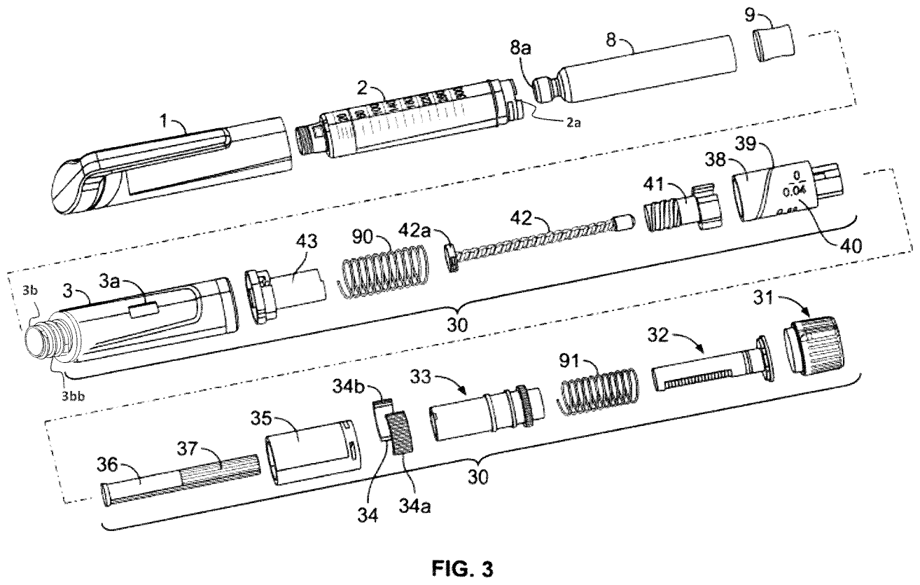

The present disclosure is applicable with a number of medicament delivery devices, for example, injection devices. One possible injection device is the pen-type design illustrated in FIGS. 1-3, where the pen-shaped injection design 10 has a dose setting mechanism 3 connected to a cartridge holder 2 holding cartridge 8 (FIG. 3) and where the cartridge holder 2 has a needle connector 7. The injection device 10 has a dose setting mechanism 30 and is illustrated in the zero-dose state as indicated by indicia 40 showing a zero through the window 3a of housing 3. Housing 3 terminates at its proximal end in a keyed connector 3b, which is illustrated as having a thread form 3bb.

FIG. 3 shows the device 10 with cap 1 removed to expose the cartridge holder 2 and the proximal needle connector 7. Pen needle 4 is typically attached to the needle connector 7 through a snap fit, thread, Luer-Lok, or other secure attachment with hub 5 such that a double ended needle cannula 6 can achieve a fluid communication with medicament contained in cartridge 8 positioned within cartridge holder 2. The cartridge 8 is sealed at the proximal end by septum 8a and with a sliding piston 9 at the opposite distal end.

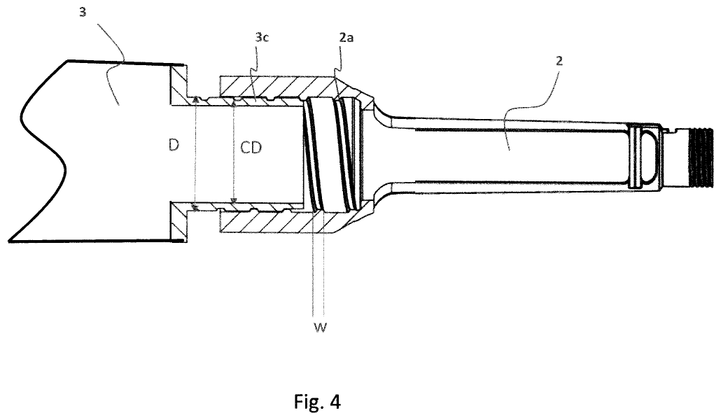

FIG. 4 illustrates an embodiment of the present disclosure where the cartridge holder 2 has a keyed connector 2a that matches the keyed connector 3c of housing 3.

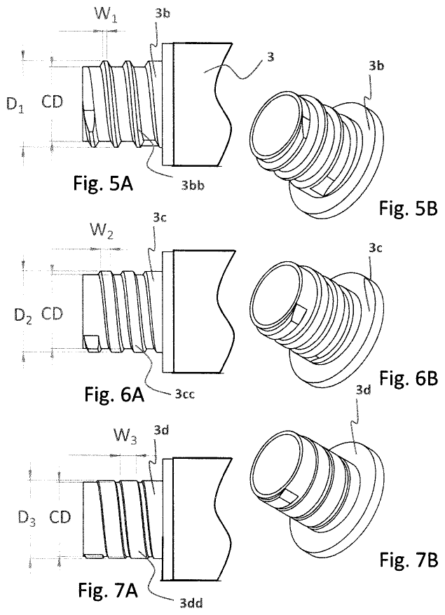

FIGS. 5A-7B illustrate an embodiment of the present disclosure where there are three different dose delivery mechanism housings 3 each of which has a different keyed connector 3b, 3c & 3d located on the proximal end of the housing 3. FIGS. 5B, 6B, and 7B show only the keyed connectors in a perspective view without showing the housing 3. Each of the keyed connectors in FIGS. 5A-7B is shown as a male connector, where each has a different thread form 3bb, 3cc, and 3dd. Each keyed connector has the same core diameter (CD) but with different outer thread diameter (D), shown as D₁, D₂ and D₃ in FIGS. 5A, 6A & 7A. Further, each of the three different keyed connectors 3b, 3c, & 3c have male thread forms having a different thread width (W), namely W₁, W₂, & W₃, where in the embodiment shown W₃>W₂>W₁. Threads 3bb, 3cc, & 3dd also each have different geometries even though each of the keyed connectors shown have the same CD. Indeed, the keyed connectors could all have the same CD, the same D, and the same W, but the thread geometries could all be different. Stated differently, a corresponding female keyed connector matching fitting with the keyed connector 3b would not allow (will prevent) attachment of a keyed connector having same CD, D and W, but having a different thread geometry.

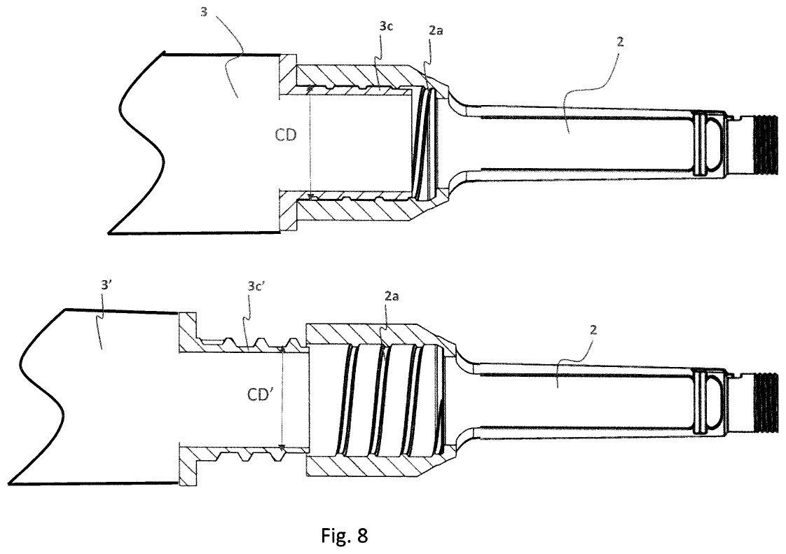

FIG. 8 shows a truncated cross-sectional side view of two identical cartridge holders 2 and two different dose setting and delivery mechanism housings 3 and 3′ each having two different keyed connectors 3c and 3c′. In the tipper illustration of FIG. 8, the keyed connectors 3c and 2a are matching, thus allowing the housing 3 and cartridge holder 2 to be connected to each other. In the bottom illustration of FIG. 8, keyed connector 3c′ is different than 3c or 2a and therefor the housing 3 cannot be connected to cartridge holder 2.

In some circumstances, manufacturing efficiency can dictate which of the keying parameters are kept constant when designing a set of keyed connectors for the medicament delivery devices. For example, it can be desirable to keep the CD the same for a set of keyed connectors and to vary only the thread geometry, D and/or W.

The present disclosure includes medicament holders, such as cartridge holder 2, having a keyed connector 2a at a distal end of the holder that matches and allows engagement to form a connection with a corresponding keyed connector, i. e., 3b, 3c or 3d. To reiterate, a corresponding keyed connector on the holder would necessarily require that the parameters of W, CD, D and the thread geometry all match with the keyed connector located on the proximal end of the dose delivery mechanism. If one of those parameters were different then the medicament holder could not be securely connected to the non-matching dose delivery mechanism.

The particular design of device 10 allows for setting of one or more of the predetermined fixed doses through the interaction of snap element 33 with dose selector 35. The rotation of the dose knob and snap element occurs during dose setting and is relative to housing 3. During the initiation of the dose delivery procedure the dose knob 31 is pressed in the proximal direction causing it and the dose selector to move axially relative to the snap element.

Part of the dose setting and delivery mechanisms of most pen-type injectors, including device 10, is a piston rod 42 as illustrated in FIG. 3. Such piston rods usually have a non-circular cross-section and have two flat surfaces that are designed to prevent the piston rod from rotating but allowing it to move linearly in the proximal direction. A nut 36 and a clutch 32 are permanently splined to each other during assembly of the dose setting mechanism through a splined connection. The splined connection ensures that clutch 32 and nut 36 are always rotationally fixed to each other during both dose setting and dose delivery. This splined connection also allows the clutch and the nut to move axially relative to each other. The sliding connection is necessary to compensate for the difference in the pitch of the thread between nut and the outer surface of the piston rod and the pitch of the thread between dose sleeve and body. The thread between driver and piston guide has basically the same pitch as the thread between piston rod and nut.

The proximal end of nut 36 has internal threads 70 that match threads 60 of piston rod 42. The distal end of clutch 32 is configured as a dose button 72 and is permanently attached to distal end of the dose knob 31 through engagement of connectors, which can also include snap locks, an adhesive and/or a sonic weld. This connection ensures that the clutch is both rotationally and axially fixed to the dose knob during both dose setting and dose delivery.

At the terminal proximal end of piston rod 42 is connector 62, shown as a snap fit, that connects with a disc or foot 42a. At the distal end of piston rod 42 is a last dose feature of the dose setting mechanism, illustrated as an enlarged section 63. This enlarge section 63 is designed to stop the rotation of nut 36 about threads 60 when the amount of medicament remaining in the cartridge 8 is less than the next highest predetermined dose setting. In other words, if the user tries to set one of the predetermined fixed dose settings that exceeds the amount of medicament remaining in the cartridge, then the enlarged section 63 will act as a hard stop preventing the nut from further rotation along threads 60 as the user attempts to reach the desired predetermined fixed dose setting.

The piston rod 42 is held in a non-rotational state relative to housing 3 during both dose setting and dose delivery because it is arranged within the non-circular pass through hole in the center of piston rod guide 43. The piston rod guide is both rotationally and axially fixed to housing 3. This fixation can be achieved when the piston rod guide is a separate component from the housing 3 as illustrated or the piston rod guide could be made integral with the housing. Piston rod guide 43 also engages the proximal end of a rotational biasing member, shown as torsion spring 90, the function of which will be explained below. This connection of the rotational biasing member to the piston rod guide anchors one end in a rotational fixed position relative to the housing.

The distal end of the rotational biasing member, for example torsion spring 90, is connected to the driver 41. Driver 41 is connected and rotationally fixed with the inner surface of dose sleeve 38 through a splined connection on the distal outer surface of the driver. On the proximal end of driver 41 on the outer surface is threads 67 that are engaged with matching threads on the inner distal surface of the piston rod guide 43. The thread between driver and piston guide has a significantly different pitch than the thread between dose sleeve and housing. The nut and the driver rotate together both during dose setting and dose cancellation and, as such, they perform essentially the same axial movement. However, this movement is independent from each other, i.e., the nut is turned by the clutch and performs an axial movement due to the thread to the piston rod, while the driver is rotated by the dose sleeve and performs an axial movement due to the thread to the piston guide. The driver is rotating during injection also, and so it actively moves in the proximal direction during injection. But, the nut does not rotate during injection and as such does not perform an active axial movement. The nut is only moving in proximal direction during injection because it is being pushed axially by the driver. The rotating driver pushing the non-rotating nut causes the injection because the piston rod is pushed forward due to the threaded engagement with the nut.

Because the torsion spring is attached to the driver 41 and the driver is rotationally fixed to the dose sleeve 38, then rotation of the dose sleeve in a first direction during dose setting will wind the torsion spring such that it exerts a counter rotational force on the dose sleeve in an opposite second direction. This counter rotational force biases the dose sleeve to rotate in a dose canceling direction.

The function of the complete injection device 10 and the dose setting mechanism according to this disclosure will now be described. Injection device 10 is provided to a user as reusable or semi-reusable device. A semi reusable means that only the dose setting and delivery mechanism 30 housed in housing 3 is reused each time a new medicament cartridge holder 3 containing a new cartridge of medicament is connected to the housing 3. A reusable device would allow reattachment of an old or previously used cartridge holder where the user has inserted a new full cartridge of medicament. A preferred configuration of the present disclosure is the semi-reusable design where each time the medicament in the cartridge is expelled or emptied, the user would be required to disconnect the cartridge holder containing the empty cartridge that is not removable from the holder. As such, the user would dispose of bother the holder and the empty cartridge together. A new cartridge holder and cartridge assembly would be connected to the housing provided that the keyed connector on the housing matches the keyed connector on the distal end of the cartridge holder.

Once the dose setting mechanism is primed, the user then selects and sets a desired fixed dose by repeating the same steps used for priming except that the dose knob will be rotated past the priming stop until the appropriate dose stop is and the desired dose value appears in the window 3a. In some cases, it is preferred to have no indicia show in the window when dialing between predetermined dose settings, while in other cases it is desirable to show an indicia in the window that is indicative of a non-settable dose position between the fixed dose settings.

Once one of the predetermined dose settings has been dialed on the dose setting mechanism, the user can then exert an axial force in the proximal direction to initiate the dose delivery procedure. The axial force exerted by the user overcomes the distally directed force exerted by the second biasing member 91 causing the dose knob 31, clutch 32 and dose selector 35 to move axially in the proximal direction relative to the snap element 33 and housing 3. This initial movement rotationally fixes the clutch and dose knob to the housing through the splined connection between the floating spline 34 and splines inside dose selector 35. The splined connection between the dose selector and floating spline 34 remains engaged during dose setting and during dose delivery even though the dose selector 35 moves axially with the dose knob 31 and relative to the floating spline 34.

As the user maintains the axial force on both the dose knob 31 and the dose button 72 during the continuation of the dose delivery procedure, the clutch 32 will abut the distal end of the snap element causing it to move axially in the proximal direction. The clutch pushes on the snap element. The snap element is fixed to the dose sleeve, so the clutch pushes on the dose sleeve. As the dose sleeve has a thread with a sufficiently high pitch relative to the body, the axial force on the dose sleeve will cause the dose sleeve and as such the snap element to turn relative to the body, and by turning relative to the body it moves in the proximal direction. The dose selector slides into the housing but does not rotate relative to the housing 3 due to the splined engagement with the housing. The rotation of the dose sleeve 38 also causes rotation of the driver 41 into the threaded connection with piston rod guide 43, which drives the piston rod proximally and results in a concurrent de-tensioning of torsion spring 90. The driver does not directly drive the piston rod. As the driver rotates, the driver moves in the proximal direction and pushes the nut forwards. As the nut doesn't turn, the driver pushes the nut and the piston rod forward.

The nut 36 does not rotate during dose delivery because of the rotationally fixed relationship with clutch 32 that is rotationally fixed to the housing through rotationally fixed relationship of the dose knob, floating spline and the housing. The nut therefore can only move axially carrying the piston rod 42 with it because the piston rod is prevented from rotating by the non-circular opening 64 engaged with the flats 203 on the piston rod. The piston rod is moved axially the same distance that the nut originally translated relative to the piston rod during dose setting. Axial movement of the piston rod causes the sliding piston 9 to also move axially relative to the inside walls of the stationary cartridge 8 forcing an amount of medicament out of the needle cannula 6 that is equivalent to the predetermined fixed dose that was set during the dose setting procedure.

If the user stops or halts the dose delivery procedure by removing the axial force on the dose knob a fail-safe mechanism is activated. Removal of the axial force causes the compression spring 91 to bias the dose knob in the distal direction. If the user halts the dose delivery between two predetermined fixed dose settings, then the dose knob and the axially fixed dose selector will both be prevented from moving proximally because of a projecting rib inside the dose selector that will stop the axially movement of dose selector and dose knob. Without this projecting rib, the dose selector would move distally such that the dose knob would re-engage with the snap element, thus placing the dose knob, clutch and nut back into rotational engagement with the snap element. The torque exerted on the snap element through the driver would then counter rotate the nut, thus reducing the set dose by an unknown amount. This counter rotation would continue until the next lowest predetermined fixed dose setting is reached, where the corresponding dose stop would stop the counter rotation. Therefore, a resumption of the halted dose delivery procedure will continue without any unknown decrease in the set dose, thus allowing the originally set predetermined dose to be delivered.

It is to be understood that the embodiments described above and shown in the drawings are to be regarded only as non-limiting examples of the possible designs of the safety assembly and such designs can be modified in many ways and still be within the scope of the present disclosure.

1. A collection of two or more dose delivery mechanisms and two or more medicament containers comprising:
a first dose delivery mechanism having a proximal end comprising a first keyed connector;
a second dose delivery mechanism having a proximal end comprising a second keyed connector;
a first holder configured to accept and hold a first medicament container containing a first medicament, a distal end of the first holder comprising a third keyed connector; and
a second holder configured to accept and hold a second medicament container containing a second medicament, where a distal end of the second holder comprises a fourth keyed connector and the second medicament is different than the first medicament,
the first keyed connector is not capable of forming a connection with the fourth keyed connector and the second keyed connector is not capable of forming a connection with the third keyed connector, and
the first keyed connector is configured to can engage and form a connection with the third keyed connector and the second keyed connector is configured to can engage and form a connection with the fourth keyed connector.

2. The collection of claim 1, wherein each of the first and second holders has an open proximal end configured to enable axial movement of a piston rod contained within one of the first or second dose delivery mechanisms such that the piston rod is configured to move beyond the proximal end into the first holder when the first holder is attached to the first dose delivery mechanism.

3. The collection of claim 2, wherein the first and second keyed connectors each comprise a thread form.

4. The collection of claim 3, wherein each of the thread forms have a same core diameter.

5. The collection of claim 3, wherein each of the thread forms have a same outer thread diameter.

6. The collection of claim 3, wherein the thread form of the first keyed connector is of opposite hand to the thread form of the second keyed connector.

7. The collection of claim 3, wherein the thread form of the first keyed connector has a first pitch and the thread form of the second keyed connector has a second pitch.

8. The collection of claim 7, wherein the first pitch and the second pitch are different.

9. The collection of claim 3, wherein the thread form of the first keyed connector has a first thread width and the thread form of the second keyed connector has a second width.

10. The collection of claim 3, wherein the thread form of the first keyed connector has a first thread geometry and the thread form of the second keyed connector has a second geometry.

11. The collection of claim 10, wherein the first thread geometry is different than the second geometry.

12. The collection of claim 1, wherein the first and second keyed connectors each comprise a male thread form having a same core diameter and different outer diameters.

13. The collection of claim 12, wherein each male thread form has a different geometry.

14. The collection of claim 12, wherein each male thread form has a different pitch.

15. The collection of claim 12, wherein the male thread form of the first keyed connector has a hand opposite that of the male thread form of the second keyed connector.

16. The collection of claim 1, wherein the first and second keyed connectors each comprise a male thread form.

17. The collection of claim 1, wherein the first and second keyed connectors each comprise a female male thread form.
